(19) United States
(12) Patent Application Publication
(10) Pub. No.: US 2006/0100566 A1
(43) Pub. Date: May 11, 2006

(54) Apparatus and method for the treatment of dysmenorrhea

(75) Inventors: Edward A. Zbella, Belleair, FL (US); Bradley S. Logan, Goodlettsville, TN (US)

Correspondence Address: HOLLAND & KNIGHT LLP, 2099 PENNSYLVANIA AVE, N.W., WASHINGTON, DC, 20006, US

(73) Assignee: Women's Medical Research Group, LLC

(21) Appl. No.: 11/019,585

(22) Filed: Dec. 22, 2004

Related U.S. Application Data
(60) Provisional application No. 60/625,703, filed on Nov. 5, 2004.

(51) Int. Cl. A61F 13/20

(52) U.S. Cl. 604/15

(57) ABSTRACT

Dysmenorrhea is characterized by painful uterine cramps during menstruation. An applicator and method are disclosed for alleviating dysmenorrhea by injecting a volume of a pharmaceutical agent into a woman's vaginal cavity. The device also functions as a tampon applicator. The device achieves both purposes by providing a cavity for the storage of the pharmaceutical agent and a hollow interior area for the storage of the tampon. The agent and tampon can be expelled together or separately from one another by way of multiple plunger elements.

### BACKGROUND OF THE INVENTION

1. Field of the Invention

This invention relates to an apparatus and method for the treatment of dysmenorrhea. More particularly, the present invention relates to an improved tampon applicator that is used in the delivery of a pharmaceutical agent to relieve uterine cramps during menstruation.

2. Description of the Background Art

Dysmenorrhea is a medical condition that afflicts many women, characterized by difficult and painful menstruation. This menstrual pain is caused by uterine contractions during which the uterus constricts either too strongly or too frequently. Either way, the result is a loss of blood to the uterus, a concurrent loss of oxygen, and pain that can sometimes be debilitating. Women suffering from dysmenorrhea frequently suffer from other conditions such as nausea, vomiting, diarrhea, and/or a generalized fatigue.

Dysmenorrhea is generally classified as either primary or secondary. Primary dysmenorrhea is more common than secondary dysmenorrhea and is characterized by the excessive production of prostaglandins, which results in painful contractions during the menstrual cycle. Prostaglandin production can be decreased by means such as non-steroidal anti-inflammatory drugs (NSAIDs), such as aspirin or Ibuprofen. Secondary dysmenorrhea usually occurs in older woman but is likewise characterized by menstrual pain. Secondary dysmenorrhea differs from primary dysmenorrhea in that it is caused by a pelvic abnormality or disease. For example, secondary dysmenorrhea can be caused by infections, adenomyosis, endometriosis, or adhesions.

Whether primary or secondary, the effects of dysmenorrhea are staggering. Approximately 40% of all adult females in the U.S. suffer from some type of menstrual pain, and 10% of all women are incapacitated by menstrual pains for up to three days each month. Dysmenorrhea is the most common reason women miss work and is the leading cause of absenteeism for women younger than thirty years. In sum, dysmenorrhea causes a tremendous disruption to the lives of those suffering from it.

There have been a variety of attempts to alleviate the effects of dysmenorrhea through the application of various pharmaceutical agents. One such example is illustrated by U.S. Pat. No. 6,086,901 to Harrison, et al. Harrison discloses a system for delivering drugs to the uterus, where a pharmaceutical agent is incorporated into one of the disclosed carriers, such that the pharmaceutical agent can be released into the vagina and delivered to the uterus by absorption via the vaginal epithelium. The drug delivery device illustrated in Harrison is generally discussed as being a tampon device, vaginal ring, pessary, tablet, suppository, or vaginal medicated tampon, with specific embodiments disclosed for suppositories and tampons. The claimed devices are impregnated with the pharmaceutical composition and positioned in the vaginal canal for delivery of the composition, although Harrison does state that delivery of the composition can be accomplished via a well on the device, or just via a lotion topically applied.

Another example is U.S. Pat. No. 4,318,405 to Sneider. Sneider discloses a tampon and drug delivery device that includes a generally cylindrical inserter tube, defining a cartridge within which a tampon is slidably received for ejection through one end of the cartridge. A cylindrical plunger member is slidably received within an opposite end of the cartridge for ejecting the tampon therefrom. The device further includes a capsule of a disintegrable material that is partially embedded in and removably held by the tampon for delivery into the vaginal cavity. All of the embodiments illustrated in Sneider involve the use of a tablet or capsule that must be disintegrated before the drug is applied.

Thus, although each of the above referenced inventions achieves its own individual objective, they both suffer from common drawbacks. Specifically, neither device allows for the injection of a pharmaceutical agent directly into a vaginal cavity by a convenient delivery device. Additionally, both inventions require increased dosages of the pharmaceutical agent by applying it inefficiently via an impregnated tampon or tampon-like device, or centrally within the vagina, or via known techniques for delivery of lotions. Accordingly, there exists a need in the art to provide an apparatus for the rapid and efficient treatment of dysmenorrhea via the direct application of appropriate dosages of suitable pharmaceutical agents to targeted regions of the vagina.

### SUMMARY OF THE INVENTION

It is therefore one of the objectives of this invention to provide a device which can deliver a volume of a pharmaceutical agent directly to an appropriate location within a vaginal cavity.

It is also an object of this invention to provide an apparatus for the insertion of a tampon into a vaginal cavity along with delivery of a suitable pharmaceutical agent.

Still another object of this invention is to provide a single apparatus that can both dispense a quantity of a pharmaceutical agent and place a tampon into a vaginal cavity.

These and other objects of the present invention are achieved by providing a system for use in treating dysmenorrhea. In the preferred embodiment described below, the system includes an applicator having an outer wall, a hollow interior, proximal and distal ends and an intermediate extent therebetween. The applicator additionally includes an applicator chamber that is formed within the outer wall of the applicator. The applicator chamber has a proximal opening for delivery of the pharmaceutical agent from the applicator chamber. The agent is dispensed via a medication plunger that is received at the proximal end of the applicator chamber. A tampon formed from an absorbent material is positioned within the hollow interior of the applicator at its distal end, with a tampon string being secured to the tampon and having a free end extending out of the proximal end of the applicator. A tampon plunger may be included, having a hollow interior and proximal and distal ends, with the distal end of the plunger being telescopically positioned within the hollow interior of the applicator. The tampon plunger thus expels the tampon from the distal end of the applicator upon insertion of the plunger into the applicator.

The foregoing has outlined rather broadly certain pertinent and important features of the present invention in order that the detailed description of the invention that follows may be better understood and its present contribution to the art more fully appreciated. Additional features of the invention are also described which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a fuller understanding of the nature and objects of the invention, reference should be had to the following detailed description taken in connection with the accompanying drawings, in which:

FIG. 1-A is a side elevational view of a first embodiment of an applicator according to the present invention.

FIG. 1-B is an end view of the applicator of FIG. 1-A illustrating the end of the applicator.

FIG. 1-C is a perspective view of the applicator, including a medication plunger and tampon plunger, which are all telescopically interrelated.

FIG. 2 is a cross-sectional view of the applicator of FIG. 1-A.

FIG. 3 is a cross-sectional view of the applicator of FIG. 1-A with the medication plunger depressed.

FIG. 4 is a cross-sectional view of the applicator of FIG. 1-A with both the medication plunger and the tampon plunger depressed.

FIG. 5 is an illustration of the applicator positioned within a vaginal cavity and with both the medication plunger and the tampon plunger depressed.

FIGS. 6-A-6-E disclose various alternative embodiments of the present invention.

Similar reference characters refer to similar parts throughout the several views of the drawings.

### DETAILED DESCRIPTION OF THE PREFERRED EMBODIMENT

The present invention relates to an apparatus and method for the treatment for dysmenorrhea. Dysmenorrhea is characterized by painful uterine cramps during menstruation. In a presently preferred embodiment, the apparatus alleviates dysmenorrhea by injecting a predetermined volume of a pharmaceutical agent into a patient's vaginal cavity. The device also functions as a tampon applicator. It thus achieves a dual purpose by providing both a cavity for the storage of a pharmaceutical agent, and a separate cavity for the storage of a tampon. The agent and tampon can be expelled together, or separately via separate plungers (e.g., expelling the agent first, then the tampon).

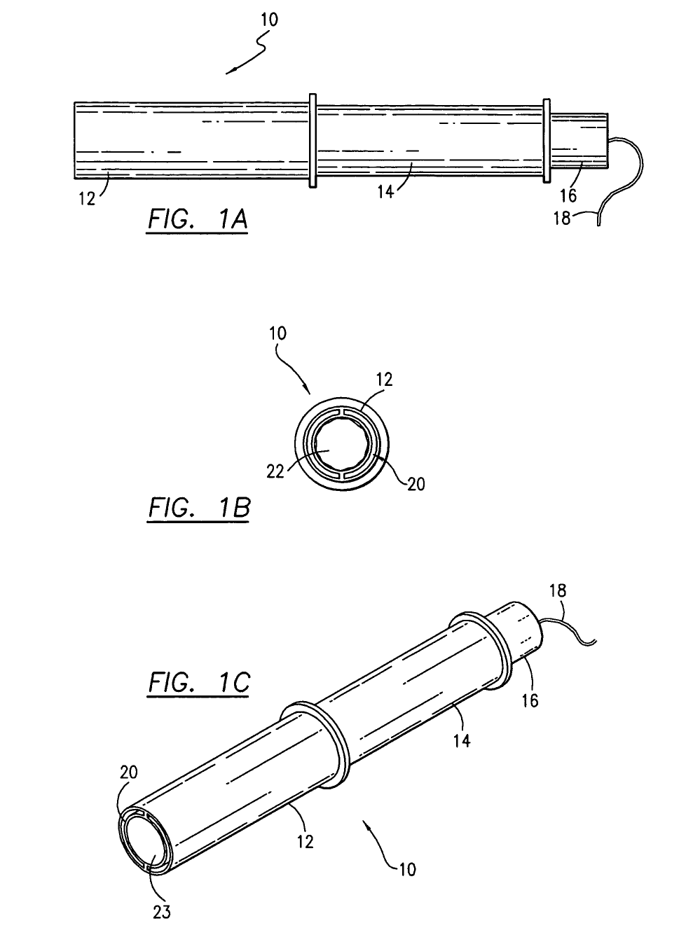

With reference now to FIG. 1-A, the various components of the presently preferred applicator 10 are illustrated. Specifically, these figures illustrate the interrelationship of the applicator 12, medication plunger 14, and tampon plunger 16. Each of these components (12, 14, 16) is telescopically related to one another and allows for the separate and sequential dispensing of a pharmaceutical compound and tampon.

The applicator tube 12 is described first in conjunction with FIGS. 1A-1C. In the preferred embodiment, applicator tube 12 has a cylindrical configuration with both proximal and distal ends (the proximal end is at the right hand side of FIG. 1A and the distal end is at the left hand side). Applicator tube 12 further includes an outer cylindrical wall that defines a hollow interior region for the placement of a tampon 22 (see FIG. 1B). Applicator tube 12 can also take the shape of a conventional tampon applicator with rounded ends to facilitate insertion into a user's vagina.

However, unlike conventional tampon applicators, applicator tube 12 of the present invention includes an applicator chamber 20 formed within the outer wall of applicator tube 12. In the illustrated embodiment, applicator tube 12 includes a double wall, with the area intermediate the double wall serving to define the cylindrical applicator chamber 20. Applicator chamber 20 includes both proximal and distal openings, with the distal opening being illustrated in FIG. 1B. FIG. 1C further illustrates the surface of the tampon 23 that is visible through the distal opening of applicator 12.

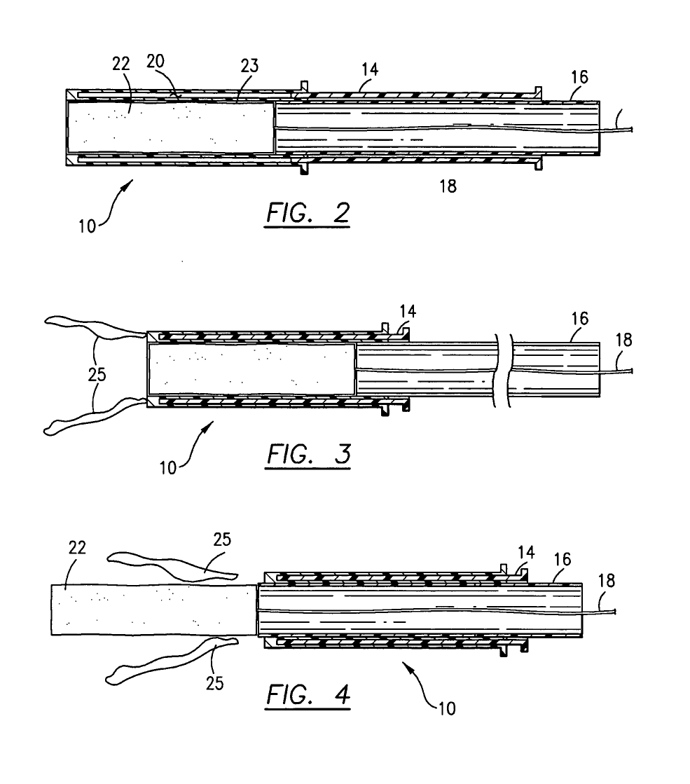

With reference now to FIGS. 2 through 4, it can be seen that applicator chamber 20 is for use in storing and dispensing a predetermined volume of a pharmaceutical agent 25. In accordance with the principals of this invention, pharmaceutical agent 25 is specifically selected for use in treating dysmenorrhea. While the pharmaceutical agent 25 may be any appropriate medicament for either treatment of the pain or the underlying disorder, common agents are selected from the group consisting of: non-steroidal anti-inflammatory drugs (NSAID); anti-prostaglandins; prostaglandin inhibitors; COX-2 inhibitors; local anesthetics; calcium channel blockers; potassium channel blockers; β-adrenergic agonists; leukotrien blocking agents; smooth muscle inhibitors; vasodilators; and drugs capable of inhibiting dyskinetic muscle contraction. Some examples of nonsteroidal anti-inflammatory drugs suitable for use include Aspirin, Ibuprofen, Indomethacin, Phenylbutazone, Bromfenac, Fenamate, Sulindac, Nabumetone, Ketorolac, and Naproxen. Examples of local anesthetics include Lidocaine, Mepivacaine, Etidocaine, Bupivacaine, 2-Chloroprocaine hydrochloride, Procaine, and Tetracaine hydrochloride. Examples of calcium channel antagonists include Diltaizem, Israpidine, Nimodipine, Felodipine, Verapamil, Nifedipine, Nicardipine, Piroxicam, and Bepridil. Examples of potassium channel blockers include Dofetilide, E-4031, Almokalant, Sematilide, Ambasilide, Azimilide, Tedisamil, RP58866, Sotalol, and Ibutilide. Examples of beta.-adrenergic agonists include Terbutaline, Salbutamol, Metaproterenol, and Ritodrine. Vasodialtors, for relieving muscle spasm, include nitroglycerin, isosorbide dinitrate and isosorbide mononitrate.

In a manner known in the art, the desired pharmaceutical agent can also be combined with a suitable biocompatible excipient (preferably with a low affinity to the active agent) for use in delivering the drug. This could include timed release forms to sustain the effective period for efficacy of the agent. The form of controlled release could be via any suitable formulation, such as bioadhesive microparticles, cream, lotion, foam, paste, ointment, or gel. Other ingredients may also be used, depending on the desired application. Such might include muco-adhesive agents (to bring the released drug in solution into prolonged contact with the mucosal surface; e.g., polymers such as an alginate, pectin, or cellulose derivative), and a penetration enhancer or sorption promoter (to enhance permeation of the drug across the uterine mucosal barrier; e.g., nonionic surface active agents, bile salts, organic solvents, and interesterified stone oil.)

The compound 25 may also come pre-loaded (e.g., for over the counter sales of applicators 10 that can be self-applied by women for common treatment formulations). They can also come empty or with just the tampon loaded, allowing a medical care provider to load an appropriate formulation and dosage into chamber 20.

As noted, the stored pharmaceutical agent or medicament 25 is delivered via a plunger. Specifically, a medication plunger 14 is included, with the plunger having a hollow interior and proximal and distal ends. The distal end of the medication plunger 14 is telescopically received within the proximal opening of applicator chamber 20. Namely, applicator chamber 20 is wide enough to accommodate medication plunger 14 such that plunger 14 is slidably positioned within chamber 20. Accordingly, as will be appreciated by those skilled in the art, as medication plunger 14 is pushed into applicator chamber 20 it functions to expel pharmaceutical agent 25 from the distal opening of chamber 20. A skilled artisan will appreciate that a variety of configurations of the plunger assembly are possible, including a fully circumferential tube that fits snuggly within chamber 20 so as to expel substantially all of the agent 25; one or more thin longitudinal members connecting a thumb projection to a distal stopper head; or even small motorized drives connected to the plunger head and controlled by any suitable button, switch or the like. In all such configurations, the applicator assembly functions to store the agent 25 while inserting the applicator 10 into the vagina, and expel substantially all of the agent 25 at the distal end of the applicator tube 12 and adjacent to the desired section of vaginal tissue for application of the agent.

Tampon 22 and tampon plunger 16 are described next in conjunction with FIGS. 2-4. Tampon 22 of the present invention is formed from a soft, porous and absorbent material that is designed to expand to absorb blood and other vaginal fluids associated with menstruation. Tampon 22 typically includes an attached tampon string 18 for removing tampon 22 from a user after use (e.g., after an appropriate time for replacing the tampon). Tampon 22 is initially positioned within the interior of applicator tube 12 towards the distal end and is subsequently expelled by way of tampon plunger 16. Tampon plunger 16 has a distal end that is telescopically positioned within both the hollow interior of applicator tube 12 and the hollow interior of medication plunger 14. Depressing tampon plunger 16 into applicator tube 12 causes it to but up against tampon 22 and force it from the applicator's opened distal end. Tampon 22 is completely expelled after tampon plunger 16 has been fully depressed into applicator 12. As noted in FIG. 4, with tampon 22 fully expelled, tampon string 18 extends out of the proximal end of applicator 12, medication plunger 14 and tampon plunger 16.

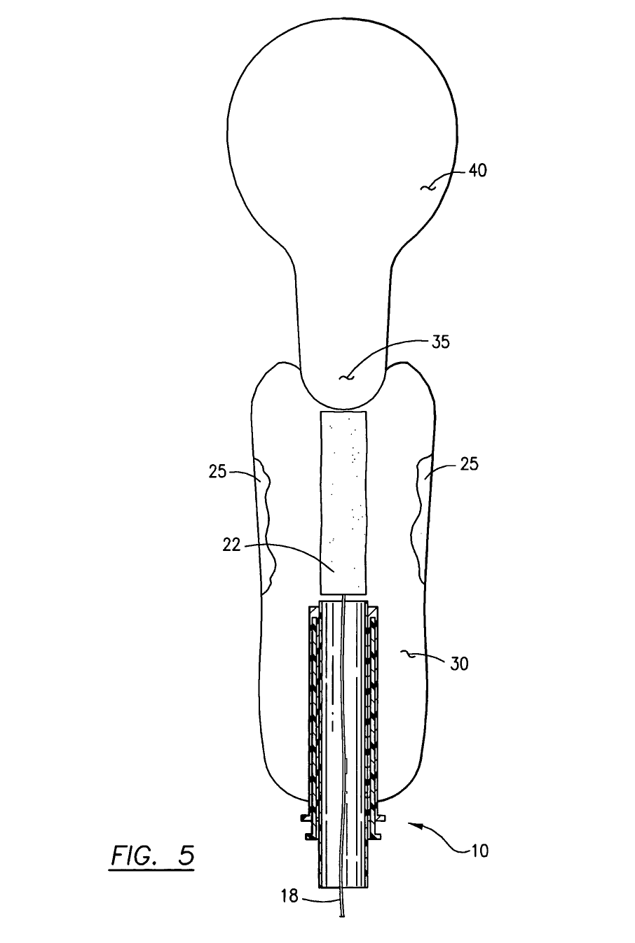

FIG. 5 illustrates the insertion of tampon 22 into the vaginal cavity 30 and adjacent the cervix 35 and uterus 40. FIG. 5 further illustrates tampon 22 inserted after pharmaceutical agent 25 has been dispensed for absorption into the mucosa along the walls of cavity 30. Alternatively, a user could simultaneously push both tampon and agent plungers (e.g., while also slowly removing the applicator 10 from the vagina), so as to rapidly position the tampon with the agent between the vaginal wall and the tampon, along a desired length of the tampon. For ease of application, applicator tube 12 is provided with protrusions or lips which can be grabbed by a users fingers, while the plunger 14 (and optionally 16) are provided with protrusions or other surfaces that can be used by the user's thumb in pushing the plungers into applicator tube 12.

In operation, applicator 10 is first readied (either pre-packaged, or by the user) with appropriate agent and tampon and fully extended—namely, with medication plunger 14 and tampon plunger 16 extended in relation to applicator 12. Apparatus 10 is then inserted into through the labia and into vaginal cavity 30. Thereafter, medication plunger 14 is depressed, whereby medication plunger 14 forces the pharmaceutical agent 25 out of the distal opening within applicator 12. This agent 25 is then absorbed into the walls of the vaginal cavity 30, either for local treatment or intravaginal delivery, e.g., to uterus 40. After pharmaceutical agent 25 has been applied, tampon 22 can be expelled from apparatus 10. This is achieved by depressing tampon plunger 16 to force tampon 22 from the end of applicator 12. The separate application of tampon 22 and pharmaceutical agent 25 insures that the medication is not inadvertently absorbed by tampon 22, which would greatly reduce the concentration of applied medication. Once tampon 22 is properly inserted, the entire apparatus 10 is removed, with only the tampon string 18 being left behind for subsequent removal of tampon 22.

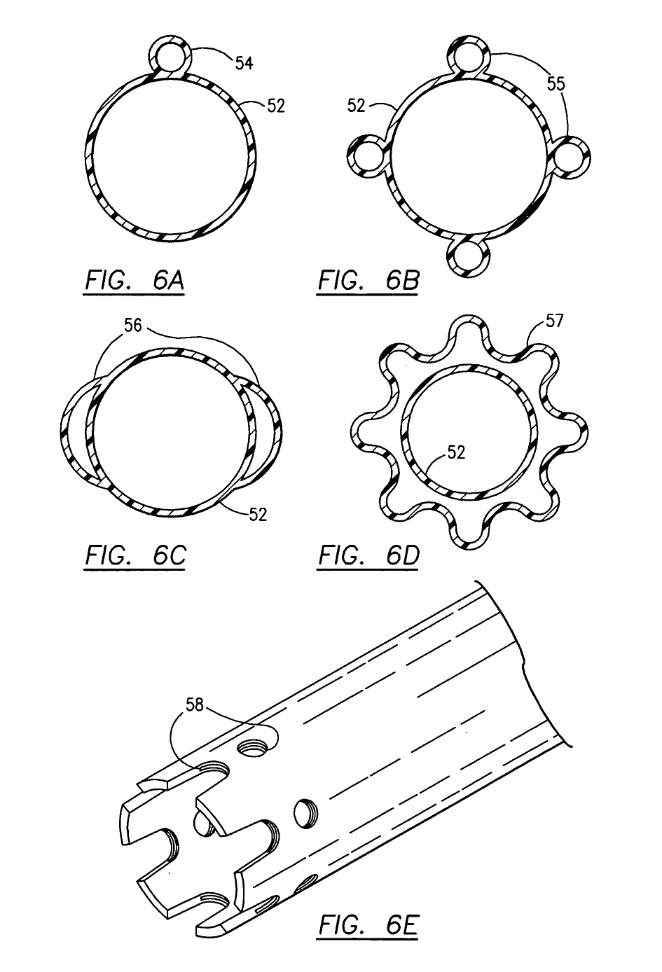

FIGS. 6A-6E illustrate various alternative arrangements for the apparatus of the present invention. Specifically, in FIG. 6A, instead of positioning the applicator chamber within the circumferential wall of the applicator, it is formed as a smaller cylindrical chamber 54 on the side of applicator 52. This embodiment employs a separate plunger (not shown) for selectively dispensing the pharmaceutical agent from the chamber 54. FIG. 6B is similar to FIG. 6A, except plural (four) applicator chambers 55 are included about the periphery of applicator 52. These could be operated by a unified plunger, or by separate plungers (e.g., to allow for more selective placement of agent along the vagina). In operation, if the cause of dysmenorrhea is localized along one section of the vagina, only those chambers that will be proximate the effected section when applicator 52 is inserted need be filled with agent.

Any of a variety of geometries for the applicator may be used, as appropriate. Thus, in the case of the embodiment of FIG. 6C, instead of being cylindrical the applicator chambers are formed as semi-circular longitudinal members 56 on lateral sides of applicator 52. The embodiment of FIG. 6D is similar in many respects to the primary embodiment; however, applicator cavity 57 takes a non-uniform shape, e.g., to accommodate a larger volume of agent and (depending on the design of the distal release slits) further distance the agent from the tampon as it is being dispensed from applicator 52.

Finally, FIG. 6E illustrates an alternative embodiment for the openings in the distal end of the applicator. Here, instead of the opening illustrated in FIG. 1B, openings 58 are formed within the interior circumference of a series of apertures and serrations. This arrangement increases the overall surface area of the openings to allow for a more rapid release of agent, and illustrates how numerous special flow patterns can be designed for dispensing the agent, based on the design choice of distal openings in the applicator.

Of course, one skilled in the art will appreciate how a variety of alternatives are possible for the individual elements, and their arrangement, described above, while still falling within the spirit of my invention. Thus, for example, other elements for placement of the operator's fingers and hand can be used for manipulating the applicator and its plungers. In addition to tampon placement, other uses such as placement of a suppository may be used, including placement in other bodily cavities (e.g., anal). While the primary use is anticipated to be placement of medicaments or pharmaceutical agents, one can readily envision the applicator being used for dispensing other liquid or semi-solid (e.g., jelly) substances about the central solid (tampon, suppository, etc.) pushed out of the applicator's hollow core. The particular structures, just like the dispensed items, will change as a matter of design choice to fit the particular applications for placement in the bodily cavity, and will also change over time as different agents and treatment regimes are developed.

While the above describes several embodiments of the invention used primarily in connection with the treatment of dysmenorrhea in conjunction with placement of tampons during menstruation, those skilled in the art will appreciate that there are a number of alternatives, based on application, system design choices and choice of regime options, that still fall within the spirit of my invention. Thus, it is to be understood that the invention is not limited to the embodiments described above, and that in light of the present disclosure, various other embodiments should be apparent to persons skilled in the art. Accordingly, it is intended that the invention not be limited to the specific illustrative embodiments, but should be interpreted within the full spirit and scope of the claims.

We claim:

1. An apparatus for use in treating dysmenorrhea comprising:
an applicator having an outer wall, a hollow interior, proximal and distal ends and an intermediate extent therebetween, an applicator chamber formed within the outer wall of the applicator, the applicator chamber having proximal and distal openings;
a pharmaceutical agent for use in treating dysmenorrhea positioned within the applicator chamber;
a medication plunger having a hollow interior, proximal and distal ends, the distal end of the medication plunger being telescopically received within the proximal opening of the applicator chamber, the medication plunger functioning to expel the pharmaceutical agent from the distal opening of the applicator chamber upon insertion of the medication plunger into the applicator chamber;
a tampon formed from an absorbent material positioned within the hollow interior of the applicator at the distal end, a tampon string secured to the tampon and having a free end extending out of the proximal end of the applicator;
a tampon plunger having a hollow interior and proximal and distal ends, the distal end of the plunger being telescopically positioned within the hollow interior of the applicator, the tampon plunger functioning to expel the tampon from the distal end of the applicator upon insertion of the plunger into the applicator.

2. An apparatus for use in treating cervical cramping comprising:
an applicator having proximal and distal ends and an intermediate extent therebetween;
an applicator chamber interconnected to the applicator, the applicator chamber having proximal and distal openings;
a pharmaceutical agent for use in treating cervical cramps positioned within the applicator chamber;
a medication plunger being telescopically received within the applicator chamber, the medication plunger functioning to expel the pharmaceutical agent from the distal opening of the applicator chamber upon insertion of the medication plunger into the applicator chamber.

3. The apparatus as described in claim 2 wherein the applicator stores a tampon that is expelled by a tampon plunger.

4. The apparatus as described in claim 2 wherein the applicator chamber is formed within a wall of the applicator.

5. The apparatus as described in claim 2 wherein the applicator chamber is formed as a separate chamber secured to an external surface of the applicator.

6. The apparatus as described in claim 2 wherein a series of applicator chambers are included and wherein each chamber stores a volume of a pharmaceutical agent.

7. A method of treating a vaginal cavity for dysmenorrhea, the method comprising the following steps:
providing an applicator, the applicator having a cavity and a hollow interior, a pharmaceutical agent for use in treating dysmenorrhea stored within the cavity, a tampon positioned within the hollow interior;
providing a medication plunger and a tampon plunger, with the medication plunger being telescopically received within the cavity of the applicator and the tampon plunger being telescopically received within the hollow interior of the applicator;
inserting the applicator into a vaginal cavity;
depressing the medication plunger into the cavity to thereby inject the pharmaceutical agent into the vaginal cavity;
thereafter depressing the tampon plunger to thereby expel the tampon from the applicator and thereby position the tampon in an area adjacent the pharmaceutical agent;
removing the applicator from the vaginal cavity.

8. A method of treating a vaginal cavity for dysmenorrhea, the method comprising the following steps:
providing an applicator, the applicator having a cavity and a hollow interior, a pharmaceutical agent for use in treating dysmenorrhea stored within the cavity;
providing a medication plunger telescopically positioned within the cavity of the applicator;
inserting the applicator into a vaginal cavity;
depressing the medication plunger into the cavity to thereby inject the pharmaceutical agent into the vaginal cavity;
removing the applicator from the vaginal cavity.

9. The method of claim 8 including the further step of inserting a tampon into the vaginal cavity after application of the pharmaceutical agent.

10. The method as described in claim 9 wherein the tampon is inserted into an area adjacent the applied pharmaceutical agent.

11. A system for placing plural differing medicinal units in a selected position within a body cavity of an animal, comprising:
an applicator having at least one wall defining an interior cavity, the cavity being configured for storing a first solid unit while inserting the applicator into the body cavity;
a first dispenser operably positioned at least partially within the interior cavity of the applicator, and operable to force the first solid unit out of the first cavity into the body cavity;
a chamber having at least one wall defining an interior chamber cavity, the chamber being connected to the applicator and positioned radially outward of the applicator interior cavity, the chamber being configured for storing a second medicinal unit of the group of semi-solids and liquids, and having a distal end configured for dispensing the second medicinal unit adjacent a wall of the body cavity; and
a second dispenser operably positioned at least partially within chamber cavity, and operable to force the second medicinal unit out of a distal end of the chamber cavity into the body cavity adjacent a wall of the body cavity.

12. The system of claim 11, wherein the first and second dispensers together form a double plunger, the applicator having a rim at a proximal end configured for gripping by a user's fingers, and the first and second dispensers each having a thumb tab at the proximal ends configured for forcing the first and second dispensers toward the proximal end of the applicator.

13. The system of claim 12, wherein the body cavity is a female vagina, the system further comprising a first solid unit positioned substantially within the first cavity and a second medicinal unit positioned within the chamber cavity, wherein the first solid unit is one of a tampon and a suppository, and the second medicinal unit is a compound comprising a pharmaceutical agent.
